SEQUENCE LISTING (12 sequences)

SEQ ID NO: 1 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 2 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

SEQ ID NO: 3 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 4 - DNA, 2207 bp, Homo sapiens
cacttcctccccagacaggggtagtgcgaggccgggcacagccttcctgtgtggttttaccgcccagagagcgtcatggacctggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctccccataggcgactcttctgcctggactagagggacctctcccagggtccctggggtggggatagggagcagatgcaatgactcaggacatccccccgccaaaagctgctcagggaaaagcagctctcccctcagagtgcaagcccctgctccagaagatagcttcaccccaatcccagctacctcaaccaatgccaaaaaaagacagggctgataagctaacaccagacagacaacactgggaaacagaggctattgtcccctaaaccaaaaactgaaagtgaaagtccagaaactgttcccacctgctggagtgaaggggccaaggagggtgagtgcaaggggcgtgggagtggcctgaagagtcctctgaatgaaccttctggcctcccacagactcaaatgctcagaccagctcttccgaaaaccaggccttatctccaagaccagagatagtggggagacttcttggcttggtgaggaaaagcggacatcagctggtcaaacaaactctctgaacccctccctccatcgttttcttcactgtcctccaagccagcgggaatggcagctgccacgccgccctaaaagcacactcatcccctcacttgccgcgtcgccctcccaggctctcaacaggggagagtgtggtgtttcctgcaggccaggccagctgcctccgcgtgatcaaagccacactctgggctccagagtggggatgacatgcactcagctcttggctccactgggatgggaggagaggacaagggaaatgtcaggggcggggagggtgacagtggccgcccaaggcccacgagcttgttctttgttctttgtcacagggactgaaaacctctcctcatgttctgctttcgattcgttaagagagcaacattttacccacacacagataaagttttcccttgaggaaacaacagctttaaaagaaaaagaaaaaaaaagtctttggtaaatggcaaaaaaaaaaaaaaaaaaaaaaaaa

SEQ ID NO: 5 - protein, 9 aa, Artificial Sequence
  CCL19 truncation mutant
GANDAEDCC

SEQ ID NO: 6 - protein, 8 aa, Artificial sequence
  CCL19 truncation mutant
ANDAEDCC

SEQ ID NO: 7 - protein, 7 aa, Artificial Sequence
  CCL19 truncation mutant
NDAEDCC

SEQ ID NO: 8 - protein, 6 aa, Artificial Sequence
  CCL19 truncation mutant
DAEDCC

SEQ ID NO: 9 - protein, 5 aa, Artificial Sequence
  CCL19 truncation mutant
AEDCC

SEQ ID NO: 10 - protein, 4 aa, Artificial Sequence
  CCL19 truncation mutant
EDCC

SEQ ID NO: 11 - protein, 3 aa, Artificial Sequence
  CCL19 truncation mutant
DCC

SEQ ID NO: 12 - protein, 2 aa, Artificial Sequence
  CCL19 truncation mutant
CC

(19) United States
(12) Patent Application Publication
(10) Pub. No.: US 2015/0307619 A1
(43) Pub. Date: Oct. 29, 2015

(54) Use of C-C Chemokine Receptor Type 7 (CCR7) Inhibitors

(71) Applicant: THE SCHEPENS EYE RESEARCH INSTITUTE, INC., Boston, MA (US)

(72) Inventors: Reza Dana, Newton, MA (US); Sunil Chauhan, Somerville, MA (US); Shilpa Kodati, Framingham, MA (US); Daniel Saban, Raleigh, NC (US)

(21) Appl. No.: 14/651,991

(22) PCT Filed: Dec. 13, 2013
(86) PCT No.: PCT/US13/75095

§ 371 Date: Jun. 12, 2015

Related U.S. Application Data
(60) Provisional application No. 61/736,976, filed on Dec. 13, 2012.

(51) Int. Cl. C07K 16/28; C07K 14/705; A61K 47/48; A61K 45/06; A61K 31/7088; A61K 39/395

(52) U.S. Cl. CPC C07K 16/2896; A61K 31/7088; A61K 39/39533; A61K 47/48384; A61K 45/06; C07K 14/70596; C07K 2317/622; C07K 2317/24; A61K 2039/505

(57) ABSTRACT

This application discloses ophthalmic formulations and methods for treating dry eye disease with a C-C chemokine receptor type 7 (CCR7) inhibitor.

### RELATED APPLICATIONS

This application claims priority to U.S. Provisional Patent Application No. 61/736,976 filed Dec. 13, 2012, the entire contents of which are hereby incorporated by reference herein.

### FEDERALLY SPONSORED RESEARCH OR DEVELOPMENT

This invention was made with Government support under Grant No. R01-EY20889 awarded by the National Institute of Health and Grant No. R01-EY021798 awarded by the National Institutes of Health. The Government has certain rights in the invention.

### FIELD OF THE DISCLOSURE

The present invention relates to compositions and methods for treating inflammatory conditions of the ocular surface.

### BACKGROUND OF THE DISCLOSURE

Dry Eye Disease (DED) is recognized as a common cause of ocular surface inflammation, and remains one of the most frequent reasons that patients seek ophthalmic care. The commonly reported symptoms of DED, which include dryness, irritation, foreign body sensation, light sensitivity, and decreased visual acuity, have a debilitating impact upon activities of daily living. DED is estimated to affect approximately 5 million Americans over the age of 50 years, with millions more experiencing intermittent symptoms of dry eye. Despite the high prevalence of this condition, current therapeutic strategies are restricted to symptomatic relief with artificial tears, as well as non-specific corticosteroid therapy and topical cyclosporine. However, the long-term use of corticosteroids is limited by the sight-threatening side effects of raised intraocular pressure and cataracts. Additionally, tolerability issues such as burning and stinging have been reported with cyclosporine. Therefore, there is an unmet need for immunomodulatory agents that target specific components of the underlying immune response in DED.

### SUMMARY OF THE DISCLOSURE

The present invention relates to pharmaceutical formulations for use in the treatment and prevention of ocular surface inflammatory diseases or disorders, e.g., dry eye disease. The invention also provides for methods for the treatment and prevention of ocular surface inflammatory disease in a subject in need of such treatment by administering the formulations of the present invention (e.g., topically or subconjunctivally) directly to the eye or region of the eye of the subject. The subject is preferably a mammal in need of such treatment, e.g., a subject that has been diagnosed with dry eye or a predisposition thereto. The mammal can be any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Compositions comprising a C-C chemokine receptor type 7 (CCR7) inhibitor are described herein. The compositions comprising a CCR7 inhibitor are for use in treating an ocular surface inflammatory disease in a subject. The composition is administered to the ocular or adnexal tissue of the subject.

A method of treating an ocular surface inflammatory disease is carried out by identifying a subject who has been diagnosed with an ocular surface inflammatory disease, and administering to an ocular or adnexal tissue a composition comprising an effective amount of a CCR7 inhibitor.

The ocular surface disease comprises dry eye disease or an autoimmune or inflammatory condition including Stevens-Johnson syndrome, and microbial keratitis. In some cases, the dry eye disease is chronic dry eye disease. The dry eye disease comprises keratoconjunctivitis sicca (KCS), Sjögren's syndrome (SS), Sjögren's syndrome associated keratoconjunctivitis sicca, non-Sjögren's syndrome associated keratoconjunctivitis sicca (e.g., as seen in graft-versus-host disease), keratitis sicca, sicca syndrome, xerophthalmia, tear dysfunction disorder, decreased tear production, aqueous tear deficiency (ATD), meibomian gland dysfunction, or hyperevaporate tear deficiency.

The CCR7 inhibitor is administered at a dose effective to reduce or prevent the induction or maintenance of pro-inflammatory T helper 1 (Th1) and/or T helper 17 (Th17) response in the draining lymphoid tissue of a subject, leading to a reduction of Th17-mediated immunity in an ocular or adnexal tissue in a subject. Additionally, the CCR7 inhibitor is administered at a dose effective to reduce or prevent migration of antigen-presenting cells to lymphoid tissue of the subject. Specifically, the CCR7 inhibitor is administered at a dose that impairs migration of antigen-presenting cells to the lymphoid tissue of the subject, thereby interfering with priming of T cells including the induction of Th1 and Th17 immunity in the draining lymph node. This is then reflected in the reduction of priming of T cells and the reduction of an observable Th1 and/or Th17 immune response in ocular or adnexal tissue.

CCR7 inhibitors/antagonists comprise a composition that inhibits or modifies the transcription, transcription stability, translation, modification, localization, secretion, or function of a polynucleotide or polypeptide encoding CCR7 or a CCR7 associated ligand, wherein the CCR7 associated ligand is CCL19 or CCL21. For example, the CCR7 inhibitor comprises an antibody conjugated directly or indirectly to a compound that inhibits or modifies the activity of CCR7.

CCR7 inhibitors/antagonists include proteins, nucleic acids, carbohydrates, antibodies, or any other molecules that decrease the activity or expression of a CCR7. For example, the CCR7 inhibitors/antagonists include any agent that prevents CCR7-mediated signal transduction. CCR7 inhibitors/antagonists also include any molecule that decreases the ocular surface expression or activity of CCR7 associated ligands CCL19 and CCL21, thereby impairing CCR7-mediated signal transduction.

The CCR7 inhibitors/antagonists are administered to the ocular surface (i.e., topically) at a dose of 1 to 2 drops. Alternatively, the CCR7 inhibitors/antagonists are subconjunctivally administered at a dose of 0.5-1 ml. The concentration of CCR7 inhibitors/antagonists is from about 0.001% to about 10% (w/v). Alternatively, the concentration of CCR7 inhibitors/antagonists is from about 0.01% to about 10% (mg/ml).

Suitable CCR7 antagonists include a neutralizing anti-CCR7 antibody, a small molecule antagonist of CCR7, a peptide that blocks CCR7, a blocking fusion protein of CCR7, and any agent directed against CCR7 ligands (e.g., an anti-CCL19 antibody or an anti-CCL21 antibody). For example, the antagonist, e.g., CCR7-specific antibody, binds to the receptor (CCR7) on a cell expressing CCR7 in an ocular tissue, thereby impairing CCR7-mediated signaling.

The neutralizing anti-CCR7 antibody is specific to CCR7, CCL19, or CCL21 in the species of the intended subject. For example, the neutralizing antibody is a monoclonal antibody, a polyclonal antibody, a single chain antibody, a humanized antibody, a recombinant antibody, or a chimeric antibody.

The pharmaceutical formulations of the present invention (e.g., CCR7 inhibitors) are formulated for ophthalmic delivery, e.g., ocular surface delivery. For example, the pharmaceutical compositions are formulated for subconjunctival administration. Alternatively, the pharmaceutical compositions are formulated for topical administration to the eye or region of the eye. For example, the formulation may comprise one or more tear substitutes. The formulation alternatively comprises an ophthalmic lubricant.

The pH of the formulation is between 5.5 and 7.5. For example, the pH of the formulation is about 7.4. The formulation is in the form of a single dose unit or in the form of a multi-dose system.

Suitable forms of the composition include a solid, a paste, an ointment, a gel, a liquid, an aerosol, a mist, a polymer, a film, an emulsion, or a suspension. In some cases, the composition is incorporated into or coated onto a contact lens. For example, the composition is a depot preparation, or a sustained-release formulation. Preferably, the formulation is an aqueous formulation. The term “aqueous” typically denotes an aqueous composition wherein the carrier is to an extent of >50%, more preferably >75% and in particular >90% by weight water.

In some cases, the method further comprises the administration of a second therapeutic agent. For example, therapeutic agents suitable for treatment of an ocular surface disease include corticosteroids, cyclosporine, or any other appropriate therapeutic agent including agents that target pathogenic cytokines in ocular surface disease such as those that target interleukin-1 (IL-1) or interleukin-17 (IL-17). Agents that target or inhibit interleukin-1 (IL-1) can inhibit or decrease IL-1α or IL-1β expression or activity. For example, agents that target or inhibit IL-1 include, but are not limited to, recombinant and/or soluble IL-1 receptor α or IL-1 receptor β, IL-1α, IL-1β, IL-1Rα, or IL-1Rβ monoclonal antibodies, and small molecule antagonists and/or inverse agonists. An example of an IL-1 receptor antagonist is Anakinra/Kineret (Amgen). Examples of agents that target or inhibit IL-17 include, but are not limited to, recombinant and/or soluble IL-17 receptors, IL-17 or IL-17 receptor monoclonal antibodies, and small molecule antagonists and/or inverse agonists. Examples of commercially available agents that target IL-17 include, but are not limited to Ixekizumab (Eli Lilly and Co.), thymoquinone (Novus Biologicals, Catalog No. NBP2-26241) and plumbagin (Novus Biologicals, Catalog No. NBP2-26242).

Alternatively, the other therapeutic agents suitable for treatment of an ocular surface disease includes agents that target inhibit tumor necrosis factor alpha (TNF-α) or matrix metalloproteinase 3 (MMP-3). Agents that target or inhibit tumor necrosis factor alpha (TNF-α) include, but are not limited to, recombinant and/or soluble TNF-α, receptors, monoclonal antibodies, and small molecule antagonists and/or inverse agonists. Examples of commercially available agents that target TNF-α, include, but are not limited to, etanerept/Embrel (Amgen, Pfizer), infliximab/Remicade (Janssen Biotech, Inc.), and adalimumab/Humira (Abbott Laboratories). Agents that target or inhibit matrix metalloproteinase-3 (MMP-3) include but are not limited to, recombinant and/or dominant negative MMP-3 mutants, monoclonal antibodies, and small molecule antagonists and/or inverse agonists. Examples of commercially available agents that target MMP-3 include, but are not limited to, MMP-3 Inhibitor I (EMD Millipore, Catalog No. 444218-5MG), MMP-3 Inhibitor IV (Santa Cruz Biotech, Catalog No. sc-311433), N-isobutyl-N(4-methoxyphenylsulfonyl)-glycylhydroxamic Acid (Calbiochem, Catalog No. 444218), N-[[(4,5-Dihydro-5-thioxo-1,3,4-thiadiazol-2-yl)amino]carbonyl]amino]-N-(cyclohexylmethyl-(2)-benzenepropanamide (Calbiochem, Catalog No. 444243).

For example, the second therapeutic agent is administered in a separate composition prior to, simultaneously with, or after administration of the CCR7 inhibitor. Alternatively, the composition comprising the CCR7 inhibitor further comprises the second therapeutic agent in the same formulation.

The composition comprising the CCR7 inhibitor is administered at a frequency that affords optimal effectiveness. For example, the composition comprising the CCR7 inhibitor is administered every 72 hours, every 48 hours, every 24 hours, every 12 hours, every 6 hours, every 3 hours, every 1 hour, or any other appropriate interval. The composition comprising the CCR7 inhibitor is administered for 1 day, 2 days, 3 days, 4 days, 7 days, 14 days, 30 days, 60 days, 90 days, or 120 days. Alternatively, the composition comprising the CCR7 inhibitor is administered for long-term use, i.e., more than 120 days, more than 150 days, more than 180 days, more than 210 days, more than 240 days, more than 270 days, more than 300 days, more than 330 days or more than 360 days.

In some cases, administration of the CCR7 inhibitor decreases expression of the pro-inflammatory cytokines tumor necrosis factor alpha (TNF-α), interleukin-17 (IL-17), IL-1α, and IL-10 that are induced in ocular surface disease. Alternatively, administration of the CCR7 inhibitor decreases expression levels of matrix metalloproteinase-3 (MMP-3) in dry eye disease.

A method of reducing or preventing migration of antigen-presenting cells (e.g., CD11b+ cells) to lymphoid tissue is carried out by administering to ocular or adnexal tissue a composition comprising a CCR7 inhibitor.

Also provided is a method of reducing or preventing the induction of pro-inflammatory T helper 1 (Tₕ1) and T helper 17 (Tₕ17)-mediated immunity in the draining lymphoid tissue of a subject with a resulting decrease in Tₕ17 immunity in an ocular or adnexal tissue in a subject, the method comprising administering to the ocular or adnexal tissue a composition comprising a CCR7 inhibitor in an amount effective to decrease the induction of dry eye disease associated pro-inflammatory Th1 and Th17 immunity in the draining lymph node and the subsequent reduction in population of Th1 and Tₕ17 cells in the ocular or adnexal tissue.

Also provided is a method of preventing ocular surface inflammatory disease comprising identifying a subject who is at risk for developing an ocular surface inflammatory disease, and administering to an ocular or adnexal tissue a composition comprising an effective amount of a C-C chemokine receptor type 7 (CCR7) inhibitor. In some cases, the subject is asymptomatic, but at high risk for developing an ocular surface inflammatory disease, e.g., post refractive surgery. The ocular surface inflammatory disease is selected from the group consisting of dry eye disease, Stevens-Johnson syndrome, and microbial keratitis.

Optionally, the method further comprises the administration of a pharmaceutically acceptable carrier. The phrase “pharmaceutically acceptable” is art-recognized and refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. Optionally, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “tear substitute” refers to molecules or compositions which lubricate, “wet,” approximate the consistency of endogenous tears, aid in natural tear build-up, or otherwise provide temporary relief of dry eye symptoms and conditions upon ocular administration.

The invention provides inhibitors of CCR7, e.g., anti-CCR7 antibodies or fragments of such antibodies, so long as they exhibit the desired biological activity.

Also included in the invention are chimeric antibodies, such as humanized antibodies. Generally, a humanized antibody has one or more amino acid residues introduced into it from a source that is non-human. Humanization can be performed, for example, using methods described in the art, by substituting at least a portion of a rodent complementarity-determining region for the corresponding regions of a human antibody.

The term “antibody” or “immunoglobulin” is intended to encompass both polyclonal and monoclonal antibodies. The preferred antibody is a monoclonal antibody reactive with the antigen. The term “antibody” is also intended to encompass mixtures of more than one antibody reactive with the antigen (e.g., a cocktail of different types of monoclonal antibodies reactive with the antigen). The term “antibody” is further intended to encompass whole antibodies, biologically functional fragments thereof, single-chain antibodies, and genetically altered antibodies such as chimeric antibodies comprising portions from more than one species, bifunctional antibodies, antibody conjugates, humanized and human antibodies. Biologically functional antibody fragments, which can also be used, are those peptide fragments derived from an antibody that are sufficient for binding to the antigen. “Antibody” as used herein is meant to include the entire antibody as well as any antibody fragments (e.g. F(ab′)₂, Fab′, Fab, Fv) capable of binding the epitope, antigen or antigenic fragment of interest.

Biologics such as polynucleotides, polypeptides (e.g., large proteins), peptides (e.g., small or medium-sized proteins), antibodies, or other biological agents are purified and/or isolated. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

“Conservatively modified variations” of a particular polynucleotide sequence refers to those polynucleotides that encode identical or essentially identical amino acid sequences, or where the polynucleotide does not encode an amino acid sequence, to essentially identical sequences. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent substitutions” or “silent variations,” which are one species of “conservatively modified variations.” Every polynucleotide sequence described herein which encodes a polypeptide also describes every possible silent variation, except where otherwise noted. Thus, silent substitutions are an implied feature of every nucleic acid sequence which encodes an amino acid. One of skill will recognize that each codon in a nucleic acid (except AUG, which is ordinarily the only codon for methionine) can be modified to yield a functionally identical molecule by standard techniques.

Similarly, “conservative amino acid substitutions,” in one or a few amino acids in an amino acid sequence are substituted with different amino acids with highly similar properties are also readily identified as being highly similar to a particular amino acid sequence, or to a particular nucleic acid sequence which encodes an amino acid. Such conservatively substituted variations of any particular sequence are a feature of the present invention. Individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art. See, e.g., Creighton (1984) Proteins, W.H. Freeman and Company, incorporated herein by reference.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide.

Although the phrase “nucleic acid molecule” primarily refers to the physical nucleic acid and the phrase “nucleic acid sequence” refers to the linear list of nucleotides of the nucleic acid molecule, the two phrases can be used interchangeably.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to reduce or prevent dry eye disease in a mammal. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, e.g., dry eye disease, so as to effect a reduction in severity and/or frequency of symptoms, eliminate the symptoms and/or their underlying cause, and/or facilitate improvement or remediation of damage.

The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

FIG. 1 is a series of photomicrographs and a bar chart illustrating the kinetics of CD11b+ APC trafficking in DED. FIG. 1A is a series of photomicrographs showing representative confocal micrographs of whole mount corneas isolated from naïve and DED mice depicting CD11b+ cell infiltration into the corneal stroma in DED (Magnification: ×400). FIG. 1B is a bar chart showing enumeration of CD11b+ cells and significantly increased numbers of CD11b+ cells in the corneal stroma of DED mice. P values have been determined using the student T test and error bars represent SEM. *, p=0.02, **, p<0.01, †, p<0.001.

FIG. 2 is a series of photomicrographs and a bar chart showing the enumeration of chemokine receptor expressing CD11b+ cells in the DED cornea. FIG. 2A is a photomicrograph showing representative confocal images of whole-mount corneas double stained with CD11b and either CCR2 or CCR5 (Magnification ×400). FIG. 2B is a bar chart showing that increased frequencies of CCR1, CCR2, CCR5, and CCR7 expressing CD11b+ cells were observed in the DED corneal stroma. P values were calculated using the student T test. Error bars represent SEM.

FIG. 3 is a bar chart showing the relative mRNA expression of chemokine ligands at the ocular surface. Real-time reverse transcriptase polymerase chain reaction (RT PCR) analysis was utilized to show a significant increase in the relative messenger RNA (mRNA) expression of CCL4 and CCL5 in conjunctival tissue from DED mice at day 12. A similar trend of elevated chemokine mRNA transcript levels was observed within the cornea of DED mice. P values were calculated using the student T test. Error bars represent SEM.

FIG. 4 is a graph with representative flow cytometric analyses showing increased frequencies of CCR7+Class II+ APCs in the draining lymph nodes (LN) of DED mice.

FIG. 5 is a line graph and a bar chart showing the effect of topical CCR7 blockade on DED severity and progression. Mice were treated topically from day 1 with anti-CCR7 antibody, isotype antibody, or remained untreated. N=6 eyes/group. P values were determined using the student T test and error bars represent SEM. FIG. 5A is a line graph showing that significantly lower clinical fluorescein scores (CFS) were observed in anti-CCR7 treated mice compared to isotype treated mice. *, p=0.01, **, p<0.001. FIG. 5B is a bar chart showing the mean percentage reduction in CFS scores (normalized to mean CFS scores in untreated DED group) in isotype and anti-CCR7 treated mice at day 8.

FIG. 6 is a schematic and a line graph showing the effect of topical CCR7 blockade on chronic DED. FIG. 6A is a schematic diagram of the experimental design to study the effect of topical CCR7 blockade on chronic DED. Mice were treated during the re-exposure period with either anti-CCR7 antibody, isotype antibody, or remained untreated. FIG. 6B is a line graph showing that significantly lower clinical fluorescein scores (CFS) were observed in anti-CCR7 treated mice compared to isotype treated mice. *, p=0.001. N=6 eyes/group. P values were calculated using the student T test and error bars represent SEM. FIG. 6C is a bar chart showing the mean percentage reduction in CFS scores (normalized to mean CFS scores in untreated DED group) in isotype and anti-CCR7 treated mice at day 29. **, p=0.0001.

FIG. 7 is a graph and a bar chart showing topical CCR7 blockade inhibits the induction of Th17 immunity in DED. FIG. 7A is a graph with representative flow cytometric plots showing decreased Th17 frequencies in the draining LN of anti-CCR7 treated mice compared with mice treated with isotype antibody and untreated DED. Values presented are the mean of three. N=3 mice per group. FIG. 7B is a bar chart showing real-time reverse transcriptase polymerase chain reaction (RT-PCR) analysis showing the fold change in IL-17a mRNA expression in conjunctiva from anti-CCR7 and isotype treated mice. Expression levels have been normalized to untreated DED as depicted by the horizontal dashed line.

FIG. 8 is a series of bar charts showing the effect of topical anti-CCR7 on the expression of DED-associated inflammatory cytokines and matrix metalloproteinases (MMP) at the ocular surface. FIG. 8A is a bar chart showing RT-PCR expression levels of MMP-3 and in corneal tissue from isotype and anti-CCR7 treated mice. *, p<0.05. FIG. 8B is a bar chart showing RT-PCR analysis of IL-1β and TNF-α, expression in corneal tissue and FIG. 8C, conjunctiva of anti-CCR7 and isotype treated mice. N=3 mice/group. Expression levels have been normalized to untreated DED as depicted by the horizontal dashed line. *, p<0.05, **, p<0.001. Data are presented as the mean+/−SEM.

### DETAILED DESCRIPTION OF THE INVENTION

Dry Eye Disease (DED) is recognized as a common cause of ocular surface inflammation, and remains one of the most frequent reasons leading patients to seek ophthalmic care (Kymionis et al., 2007 Am J Ophthalmol, 143:409-15). The commonly reported symptoms of DED, which include dryness, irritation, foreign body sensation, light sensitivity, and decreased visual acuity, can have a debilitating impact upon activities of daily living. The disease is estimated to affect approximately 5 million Americans over the age of 50 years, with millions more experiencing intermittent symptoms of dry eye (Asbell and Spiegel, 2007 Ocul Surf, 5:93-107).

Dry eye disease is a highly prevalent condition, estimated to affect 10-20% of the adult population (Zhu et al., 2007 Ocul Surf, 5(2): 75-92). Despite the high prevalence of dry eye disease, current therapy for moderate to severe dry eye disease relies on nonspecific anti-inflammatory agents, such as corticosteroids, which are fraught with side effects. Specifically, current therapeutic strategies are restricted to symptomatic relief with artificial tears, as well as non-specific corticosteroid therapy and topical cyclosporine (Restasis). However, the long-term usage of corticosteroids is limited by the sight-threatening side effects of raised intraocular pressure and cataracts (Kyrieleis et al., 2000 Curr Opin Ophthalmol, 11:478-483). Additionally, tolerability issues such as burning and stinging have been reported with Restasis® (cyclosporine; Brown et al., 2009 Arch Ophthalmol, 127:146-152). Therefore, given the nonspecific nature and adverse side effect profile of these current therapies, there remains an unmet need for immunomodulatory agents that focus on targeting specific components of the underlying immune response in DED.

### Diagnosis of Dry Eye Disease

Dry eye disease is characterized by symptoms, ocular surface damage, reduced tear film stability, tear hyperosmolarity, and inflammatory components. Dry eye disease can be diagnosed through a variety of tests (symptom questionnaires, ocular surface staining, tear break-up time, and osmometry) (See, e.g., Bron A J, 2001 Sury Ophthalmol, 45 Suppl 2:S221-6, incorporated herein by reference). According to Khanal et al., tear osmolarity is the best single test for the diagnosis of dry eye, whereas a battery of tests employing a weighted comparison of tear turnover rate (TTR), evaporation, and osmolarity measurements derived from discriminant function analysis is the most effective. (Khanal et al., 2008 Invest Ophthalmol Vis Sci, 49: 1407-1414, incorporated herein by reference). Dry eye disease symptoms also includes symptoms associated with eyelid margin and/or the meibomian glands, including thickening, itchiness, redness or swelling of the eyelids; blocked meibomian glands or abnormal meibomian gland secretions.

### CCR7 Antagonist(s)

C-C chemokine receptor type 7 (CCR7) is a protein (Genbank Accession No. NP_{—}001829.1 (GI:4502641), incorporated herein by reference) that in humans is encoded by the CCR7 gene (Genbank Accession No. NM_{—}001838.3 (GI:299473754), incorporated herein by reference). CCR7 has also recently been designated CD197 (cluster of differentiation 197).

The human amino acid sequence for CCR7 is shown below (Genbank Accession No. NP_{—}001829.1 (GI:4502641): (SEQ ID NO: 3)

MDLGKPMKSVLVVALLVIFQVCLCQDEVTDDYIGDNTTVDYTLFESLCSKK
DVRNFKAWFLPIMYSIICFVGLLGNGLVVLTYIYFKRLKTMTDTYLLNLAV
ADILFLLTLPFWAYSAAKSWVFGVHFCKLIFAIYKMSFFSGMLLLLCISID
RYVAIVQAVSAHRHRARVLLISKLSCVGIWILATVLSIPELLYSDLQRSSS
EQAMRCSLITEHVEAFITIQVAQMVIGFLVPLLAMSFCYLVIIRTLLQARN
FERNKAIKVIIAVVVVFIVFQLPYNGVVLAQTVANFNITSSTCELSKQLNI
AYDVTYSLACVRCCVNPFLYAFIGVKFRNDLFKLFKDLGCLSQEQLRQWSS
CRHIRRSSMSVEAETTTTFSP

The human nucleic acid sequence for CCR7 is shown below (Genbank Accession No. NM_{—}001838.3 (GI:299473754): (SEQ ID NO: 4)

CACTTCCTCCCCAGACAGGGGTAGTGCGAGGCCGGGCACAGCCTTCCTGTGTGGTTTTACCGCCCAGAGA
GCGTCATGGACCTGGGGAAACCAATGAAAAGCGTGCTGGTGGTGGCTCTCCTTGTCATTTTCCAGGTATG
CCTGTGTCAAGATGAGGTCACGGACGATTACATCGGAGACAACACCACAGTGGACTACACTTTGTTCGAG
TCTTTGTGCTCCAAGAAGGACGTGCGGAACTTTAAAGCCTGGTTCCTCCCTATCATGTACTCCATCATTT
GTTTCGTGGGCCTACTGGGCAATGGGCTGGTCGTGTTGACCTATATCTATTTCAAGAGGCTCAAGACCAT
GACCGATACCTACCTGCTCAACCTGGCGGTGGCAGACATCCTCTTCCTCCTGACCCTTCCCTTCTGGGCC
TACAGCGCGGCCAAGTCCTGGGTCTTCGGTGTCCACTTTTGCAAGCTCATCTTTGCCATCTACAAGATGA
GCTTCTTCAGTGGCATGCTCCTACTTCTTTGCATCAGCATTGACCGCTACGTGGCCATCGTCCAGGCTGT
CTCAGCTCACCGCCACCGTGCCCGCGTCCTTCTCATCAGCAAGCTGTCCTGTGTGGGCATCTGGATACTA
GCCACAGTGCTCTCCATCCCAGAGCTCCTGTACAGTGACCTCCAGAGGAGCAGCAGTGAGCAAGCGATGC
GATGCTCTCTCATCACAGAGCATGTGGAGGCCTTTATCACCATCCAGGTGGCCCAGATGGTGATCGGCTT
TCTGGTCCCCCTGCTGGCCATGAGCTTCTGTTACCTTGTCATCATCCGCACCCTGCTCCAGGCACGCAAC
TTTGAGCGCAACAAGGCCATCAAGGTGATCATCGCTGTGGTCGTGGTCTTCATAGTCTTCCAGCTGCCCT
ACAATGGGGTGGTCCTGGCCCAGACGGTGGCCAACTTCAACATCACCAGTAGCACCTGTGAGCTCAGTAA
GCAACTCAACATCGCCTACGACGTCACCTACAGCCTGGCCTGCGTCCGCTGCTGCGTCAACCCTTTCTTG
TACGCCTTCATCGGCGTCAAGTTCCGCAACGATCTCTTCAAGCTCTTCAAGGACCTGGGCTGCCTCAGCC
AGGAGCAGCTCCGGCAGTGGTCTTCCTGTCGGCACATCCGGCGCTCCTCCATGAGTGTGGAGGCCGAGAC
CACCACCACCTTCTCCCCATAGGCGACTCTTCTGCCTGGACTAGAGGGACCTCTCCCAGGGTCCCTGGGG
TGGGGATAGGGAGCAGATGCAATGACTCAGGACATCCCCCCGCCAAAAGCTGCTCAGGGAAAAGCAGCTC
TCCCCTCAGAGTGCAAGCCCCTGCTCCAGAAGATAGCTTCACCCCAATCCCAGCTACCTCAACCAATGCC
AAAAAAAGACAGGGCTGATAAGCTAACACCAGACAGACAACACTGGGAAACAGAGGCTATTGTCCCCTAA
ACCAAAAACTGAAAGTGAAAGTCCAGAAACTGTTCCCACCTGCTGGAGTGAAGGGGCCAAGGAGGGTGAG
TGCAAGGGGCGTGGGAGTGGCCTGAAGAGTCCTCTGAATGAACCTTCTGGCCTCCCACAGACTCAAATGC
TCAGACCAGCTCTTCCGAAAACCAGGCCTTATCTCCAAGACCAGAGATAGTGGGGAGACTTCTTGGCTTG
GTGAGGAAAAGCGGACATCAGCTGGTCAAACAAACTCTCTGAACCCCTCCCTCCATCGTTTTCTTCACTG
TCCTCCAAGCCAGCGGGAATGGCAGCTGCCACGCCGCCCTAAAAGCACACTCATCCCCTCACTTGCCGCG
TCGCCCTCCCAGGCTCTCAACAGGGGAGAGTGTGGTGTTTCCTGCAGGCCAGGCCAGCTGCCTCCGCGTG
ATCAAAGCCACACTCTGGGCTCCAGAGTGGGGATGACATGCACTCAGCTCTTGGCTCCACTGGGATGGGA
GGAGAGGACAAGGGAAATGTCAGGGGCGGGGAGGGTGACAGTGGCCGCCCAAGGCCCACGAGCTTGTTCT
TTGTTCTTTGTCACAGGGACTGAAAACCTCTCCTCATGTTCTGCTTTCGATTCGTTAAGAGAGCAACATT
TTACCCACACACAGATAAAGTTTTCCCTTGAGGAAACAACAGCTTTAAAAGAAAAAGAAAAAAAAAGTCT
TTGGTAAATGGCAAAAAAAAAAAAAAAAAAAAAAAAA

The present invention discloses a method for treatment of inflammatory conditions of the ocular surface, including dry eye disease, comprising ocular surface delivery (e.g., topical or subconjunctival administration) of a CCR7 antagonist(s), in combination with either a pharmaceutically suitable vehicle and/or another therapeutic agent. A CCR7 antagonist comprises any agent able to prevent CCR7 mediated signal transduction in cells, and may include, without limitation, a blocking fusion molecule or antibody directed against CCL19 and CCL21 ligands, neutralizing anti-CCR7 antibody, soluble peptides able to bind CCR7, a blocking fusion protein or antibody against CCR7, and small molecule antagonist of CCR7.

Antagonists may include proteins, nucleic acids, carbohydrates, antibodies, or any other molecules that decrease the effect of a protein. As used herein, antagonists of CCR7 include any compound (agent) which modulates functions of CCR7, such as a protein, peptide, small organic molecule, nucleic acid, peptidomimetic, soluble chemokine receptor, and antibody.

For example, antagonists of CCR7 include an antibody which binds to CCR7 and inhibits the interaction between CCR7 and a chemokine (or ligand for CCR7), an agent (e.g., a fragment of CCR7) which binds to the chemokine receptor but does not elicit intracellular signaling events, and a compound which reduces or inhibits the CCR7 expression. Similarly, exemplary antagonists of a chemokine receptor includes an antibody which binds to the chemokine receptor and inhibits the interaction between the chemokine ligand and CCR7, an agent (e.g., a fragment of the chemokine receptor) which binds to CCR7 and prevents the interaction between CCR7 and the wild-type chemokine ligand, and a compound which reduces or inhibits the chemokine receptor expression.

Antibodies are exemplary antagonists. Antibodies may be polyclonal or monoclonal; intact or truncated, e.g., F(ab′)2, Fab, Fv; xenogeneic, allogeneic, syngeneic, or modified forms thereof, e.g., humanized, chimeric, etc. Preferably, the antibody agonist is a neutralizing antibody (i.e., an antibody whose binding does not lead to the lysis or destruction of the CCR7 expressing cell).

Antibody generation against CCR7 polypeptide can be obtained by administering the polypeptide or epitope-bearing fragments, analogs or cells to an animal, preferably a nonhuman, using routine protocols. For preparation of monoclonal antibodies, any technique which provides antibodies produced by continuous cell line cultures can be used. Examples include the hybridoma technique (Kohler, et al., Nature (1975) 256:495-497), the trioma technique, the human B-cell hybridoma technique (Kozbor, et al., Immunology Today (1983) 4:72), and the EBV-hybridoma technique (Cole, et al., Monoclonal Antibodies And Cancer Therapy, pp. 77-96, Alan R. Liss, Inc., 1985). Techniques for the production of single chain antibodies (U.S. Pat. No. 4,946,778) can also be adapted to produce single chain antibodies (e.g., against CCR7). Also, transgenic mice or other organisms including other mammals, may be used to express humanized antibodies.

The neutralizing anti-CCR7 antibody has a binding specificity to CCR7, CCL19, or CCL21 in the species of the subject. For example, the anti-CCR7 antibody has a binding specificity of mouse CCR7 (R&D, Catalog No.: MAB3477)

Potential antagonists may include a small molecule (such as a peptidomimetic) that binds to CCR7, making it either more readily accessible or inaccessible to the other binding partner such that normal biological activity is enhanced or prevented. Examples of small molecules include, but are not limited to, small peptides or peptide-like molecules (e.g., a peptidomimetic). As used herein, the term “peptidomimetic” includes chemically modified peptides and peptide-like molecules that contain non-naturally occurring amino acids, peptoids, and the like. The term includes compounds that are partially amino acid (peptidic) in nature and partially organic chemical in nature. For example, at least one peptide bond is replaced by another more durable bond or by an organic molecule having the ability to retain the functionality of amino acids they replace. Peptidomimetics also include molecules bearing identifiable resemblance to a peptide that, as a ligand of a biological receptor, can imitate or inhibit the effect of a natural peptide. A “pseudopeptide” comprises a peptidomimetic where one or more peptide bonds have been replaced with an isostere, a surrogate functionality that is isosteric and/or isoelectronic with a peptide amide bond.

Peptidomimetics provide various advantages over a peptide, including enhanced stability when administered to a subject. The peptidomimetic has at least 10% of the activity of the reference peptide/polypeptide, e.g., at least 20%, at least 30%, at least 40%, at least 50%, at least 60%, at least 70%, at least 80%, at least 90%, at least 99%, or 100% of the activity of the reference peptide/polypeptide. Methods for identifying a peptidomimetic are well known in the art and include the screening of databases that contain libraries of potential peptidomimetics. For example, the Cambridge Structural Database contains a collection of greater than 300,000 compounds that have known crystal structures (Allen et al., Acta Crystallogr. Section B, 35:2331 (1979)). Where no crystal structure of a target molecule is available, a structure can be generated using, for example, the program CONCORD (Rusinko et al., J. Chem. Inf. Comput. Sci. 29:251 (1989)). Another database, the Available Chemicals Directory (Molecular Design Limited, Informations Systems; San Leandro Calif.), contains about 100,000 compounds that are commercially available and also can be searched to identify potential peptidomimetics of CCR7.

In particular, potential antagonists also include soluble forms of a chemokine receptor (e.g., CCR7), such as fragments of the receptor which bind to CCR7 and prevent CCR7 from interacting with membrane bound (wild-type) chemokine receptor. Optionally, the fragments are derived from the intracellular or extracellular domains of CCR7. See, e.g., U.S. Publication No. 2011/0014128, incorporated by reference in its entirety.

Antagonists also encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl, sulfhydryl or carboxyl group. The CCR7 antagonist may be a thiadiazoledioxides and thiadiazoleoxides. See e.g., U.S. Pat. No. 7,691,856, incorporated herein by reference in its entirety by reference in its entirety. The CCR7 antagonist may be a tertiary amine containing a multiplicity of heteroaromatic substituents as described in U.S. Pat. No. 6,835,731 and U.S. Pat. No. 6,864,265, incorporated herein by reference in their entireties. The CCR7 antagonist may be a piperazinylpiperidine derivative as described in U.S. Pat. No. 7,678,798, incorporated herein by reference in its entirety.

Candidate antagonists can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant, and animal extracts are available or readily produced. Additionally, natural or synthetically produced libraries and compounds can be modified through conventional chemical, physical, and biochemical means. Known pharmacological agents may be subjected to directed or random chemical modifications, such as acylation, alkylation, esterification, and amidification, to produce structural analogs.

### CCL19 and CCL21 Ligands

Chemokine (C-C motif) ligand 19 (CCL19) and Chemokine (C-C motif) ligand 21 (CCL21) are CCR7 ligands. CCL19 is a small cytokine belonging to the CC chemokine family that is also known as EBI1 ligand chemokine (ELC) and macrophage inflammatory protein-3-beta (MIP-3-beta). CCL19 is expressed abundantly in thymus and lymph nodes, with moderate levels in trachea and colon and low levels in stomach, small intestine, lung, kidney and spleen. This chemokine elicits its effects on its target cells by binding to the chemokine receptor chemokine receptor CCR7. CCL19 attracts certain cells of the immune system, including antigen-presenting cells and antigen-engaged B cells, CCR7+ central-memory T-Cells. Human CCL19 is a 98 amino acid protein having the following sequence:

(SEQ ID NO: 1)
malllalsll vlwtspaptl sgtndaedcc lsvtqkpipg
yivrnfhyll ikdgcrvpav vfttlrgrql cappdqpwve
riiqrlqrts akmkrrss

Chemokine (C-C motif) ligand 21 (CCL21) is a small cytokine belonging to the CC chemokine family. This chemokine is also known as 6Ckine (because it has six conserved cysteine residues instead of the four cysteines typical to chemokines), exodus-2, and secondary lymphoid-tissue chemokine (SLC). The gene for CCL21 is located on human chromosome 9. CCL21 elicits its effects by binding to a cell surface chemokine receptor known as CCR7. Human CCL21 is an 134 amino acid protein having the following sequence:

(SEQ ID NO: 2)
maqslalsll ilvlafgipr tqgsdggaqd cclkysqrki
pakvvrsyrk qepslgcsip ailflprkrs qaelcadpke
lwvqqlmqhl dktpspqkpa qgcrkdrgas ktgkkgkgsk
gckrtersqt pkgp

N-terminal truncation mutants of CCL19 and CCL21 may be used as antagonists to CCR7. See, e.g., Pilkington et al, J Biol Chem. 2004 Sep. 24; 279(39):40276-82, incorporated herein by reference in its entirety. As described in Pilkington, N-terminal truncation mutants of CCL21 not only inhibit CCL21-mediated chemotaxis but also CCL19-mediated chemotaxis. Examples of N-terminal CCL19 mutants, which function as CCR7 antagonists, are described in Pilkington, and set forth below. Only the first 2-8 amino acids of each mutant are depicted below to demonstrate the truncations at the N terminus with respect to the first 9 amino acids of the wild-type CCL19.

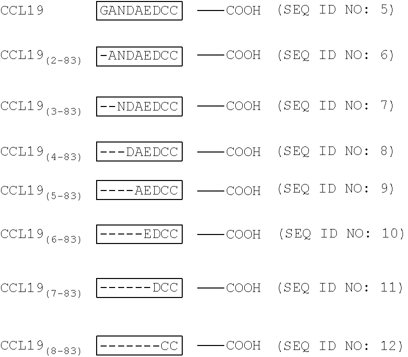

For example, the CCR7 antagonist(s) comprise an N-terminal truncation mutants of CCL19 and CCL21 wherein 1 to 25 of the N-terminal amino acids have been deleted. This includes, for example, N-terminal truncation mutants of CCL19 and CCL21 wherein the N-terminal amino acids 1 to 2, 1 to 3, 1 to 4, 1 to 5, 1 to 6, 1 to 7, 1 to 8, 1 to 9, 1 to 10, 1 to 11, 1 to 12, 1 to 13, 1 to 14, 1 to 15, 1 to 16, 1 to 17, 1 to 18, 1 to 18, 1 to 19, 1 to 20, 1 to 21, 1 to 22, 1 to 23, 1 to 24, 1 to 25 have been deleted. The N-terminal truncation mutants of CCL19 and CCL21 may have sequence identity with the corresponding amino acids of SEQ ID NO: 1 and SEQ ID NO: 2 that is at least 80%, at least 85%, at least 90%, at least 95%, at least 97%, or at least 98%, such that the N-terminal truncation mutants retain CCR7 antagonism.

### Methods of Use

The topical ophthalmic formulations of the present invention are useful to treat inflammatory conditions of the ocular surface, e.g., dry eye disease. Thus, the invention also provides methods for the treatment of inflammatory conditions of the ocular surface in a subject in need of such treatment by administering the ophthalmic formulations of the present invention directly to the eye or region of the eye of the subject.

Pharmaceutical formulations comprising at least one CCR7 antagonist of the invention may be used for the treatment inflammatory conditions of the ocular surface. For example, the pharmaceutical compositions are formulated for topical administration to the eye (e.g., subconjunctival administration; eye drops). Optionally, the pharmaceutical compositions may further comprise a tear substitute.

Also provided are methods for treating inflammatory conditions of the ocular surface in a subject in need thereof comprising administering to the eye surface of the subject a pharmaceutical composition comprising an effective amount of at least one (e.g., 1, 2, 3, 4, 5, 6, 7, 8, etc.) CCR7 antagonist(s). Optionally, the administration of CCR7 antagonist(s) to the eye of a subject in need of treatment of inflammatory conditions of the ocular surface is also effective to mitigate or reduce one or more symptoms associated with a disease or condition of inflammatory conditions of the ocular surface. The subject is preferably a human, but may be another mammal, for example a dog, a cat, a rabbit, a mouse, a rat, or a non-human primate.

The formulations of the present invention contain an amount of CCR7 antagonist(s), and optionally one or more additional active ingredients, that is effective for the intended use. Particular dosages are also selected based on a number of factors including the age, sex, species and condition of the subject. Effective amounts can also be extrapolated from dose-response curves derived from in vitro test systems or from animal models. The term “effective amount” means an amount of CCR7 antagonist(s) that is sufficient to eliminate, reduce or maintain (e.g., prevent the spread of) a symptom as a result of an inflammatory condition of the ocular surface. The effective amount is the amount sufficient for the treatment or prevention of an inflammatory condition of the ocular surface. “Treatment” in this context refers to reducing or ameliorating at least one symptom as a result of an inflammatory condition of the ocular surface. “Prevention” in this context refers to a reduction in the frequency of, or a delay in the onset of, symptoms associated with a disease or condition, relative to a subject who does not receive the composition. The invention features methods of treating inflammatory conditions of the ocular surface in a subject comprising use of the formulations described above. For example, a method of treating inflammatory conditions of the ocular surface may comprise administering to the eye surface of the subject a pharmaceutical composition comprising an effective amount of at least one CCR7 antagonist and a tear substitute in a pharmaceutically acceptable carrier.

### Ophthalmic Formulations

The differences in the intended clinical usage of the CCR7 antagonist/inhibitor in ocular surface/DED compared with ocular allergy are underpinned by the differences in the pathogenesis of the two conditions. DED is driven by a Th17 and Th1 (T helper-1 cells characterized by the secretion of interferon-gamma) mediated response. The goal of treatment with the CCR7 antagonist/inhibitor is to impair the induction and maintenance of this Th17 and Th1 response. By contrast, the principle T cells involved in ocular allergy are Th2 (T helper-2 cells). However, both conditions comprise a chronic component.

Antagonists may be formulated in combination with a suitable pharmaceutical carrier. Such formulations comprise a therapeutically effective amount of the antagonist, and a pharmaceutically acceptable carrier (excipient). Such carriers include, but are not limited to, saline, buffered saline, dextrose, water, glycerol, ethanol, and combinations thereof. Formulation should suit the mode of administration, and is well within the skill of the art.

For example, the pharmaceutical compositions of the invention may comprise combinations of at least one (e.g., 1, 2, 3, 4, 5, 6, etc.) CCR7 antagonist(s). In one aspect, the pharmaceutical compositions are formulated for subconjunctival administration. For example, the pharmaceutical compositions are formulated for topical administration to the eye (e.g., subconjunctival administration; eye drops). The pharmaceutical compositions may further comprise a tear substitute.

The concentration of CCR7 antagonist(s) are from 0.001% to 10.0% (w/v), e.g., 0.01% to 9.9%, 1% to 9%, 2% to 8%, 3% to 7%, 4% to 6% or about 5%. Alternatively, the concentration of CCR7 antagonist(s) are from 0.001% to 5%, 0.001% to 2%, 0.001% to 1%, 0.001% to 0.5%, 0.001% to 0.1%, 0.001% to 0.05%, 0.001% to 0.01%, 0.01% to 1%, 0.01% to 5%, 0.01% to 2%, 0.01% to 1%, 0.01% to 0.5%, 0.01% to 1%, 0.1% to 5%, 0.1% to 2%, 0.1% to 1%, 0.5% to 5%, 0.5% to 2%, or 0.5% to 1%, wherein the ranges are inclusive of the lower and upper limit.

Preferably, the pharmaceutical compositions according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

Any of a variety of carriers may be used in the formulations of the present invention including water, mixtures of water and water-miscible solvents, such as C1- to C7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient. Additional ingredients that may be included in the formulation include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 10 percent by weight.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, Nal, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9%±0.1% solution of sodium chloride or a 2.5%±0.3% solution of glycerol. An osmolality of about 225 to 400 mOsm/kg is preferred, more preferably 280 to 320 mOsm.

The at least one CCR7 antagonist(s) may be administered by the use of or in the form of hydrogels, drug-eluting contact lenses, and nanosystems (liposomal systems, dendrimers, solid biodegradable nanoparticles, nanogels), depot delivery (or sustained-release) systems, and/or irrigating solutions.

Ophthalmic formulations, eye ointments, creams, salves, powders, solutions and the like, are also contemplated as being within the scope of this invention.

### Eye Drops

The use of CCR7 antagonist(s) in the eyedrop mode for treatment of inflammatory conditions of the ocular surface and dry eye disease will enhance their effect by alleviating the bioavailability issue seen in systemic administration.

The eye drop may be formulated with or without one or more tear substitutes. Also provided are pharmaceutical compositions comprising an effective amount of one or more (e.g., 1, 2, 3, 4, 5, 6, 7, 8, 9, etc.) CCR7 antagonist(s) and a tear substitute in a pharmaceutically acceptable carrier for the treatment of inflammatory conditions of the ocular surface. The CCR7 antagonist(s) and tear substitute may act synergistically to provide a longer dwell time of the CCR7 antagonist(s) on the ocular surface, thus increasing duration and efficacy of action.

A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include, but are not limited to cellulose esters such as Bion Tears®, Celluvisc®, Genteal®, OccuCoat®, Refresh®, Teargen II®, Tears Naturale®, Tears Natural II®, Tears Naturale Free®, and TheraTears®; and polyvinyl alcohols such as Akwa Tears®, HypoTears®, Moisture Eyes®, Murine Lubricating®, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®.

In one aspect, the tear substitute contains hydroxypropylmethylcellulose. The tear substitute is Genteal® lubricating eye drops. GenTeal® (CibaVision—Novartis) is a sterile lubricant eye drop containing hydroxypropyl methylcellulose 3 mg/g and preserved with sodium perborate.

The pharmaceutical compositions of the invention may comprise combinations of one or more CCR7 antagonist(s) and one or more tear substitutes.

Preferably, one to two drops of the eyedrop formulation is administered to the subject. The eyedrop formulation is administered to the subject once or multiple times a day.

### Therapeutic Administration

The effective amount of the active agents in the formulation will depend on absorption, inactivation, and excretion rates of the drug as well as the delivery rate of the compound from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the active agent and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

The ophthalmic formulations containing and effective amount of CCR7 antagonist are administered to a subject once a day, twice a day, three times a day, or four times a day. For example, in an eyedrop formulation, 1 to 2 drops are administered once, twice, three times, or four times a day.

The combined use of several active agents formulated into the compositions of the present invention may reduce the required dosage for any individual component because the onset and duration of effect of the different components may be complimentary. In such combined therapy, the different active agents may be delivered together or separately, and simultaneously or at different times within the day.

### Packaging

The formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

### Kits

This invention provides kits for the packaging and/or storage and/or use of the formulations described herein, as well as kits for the practice of the methods described herein. Thus, for example, kits may comprise one or more containers containing one or more ophthalmic solutions, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

### Example 1

### Corneal CD11b⁺ Antigen-Presenting Cells Up-Regulate Chemokine Receptor Expression in DED

The normal cornea is endowed with a heterogeneous population of antigen-presenting cells (APCs) including CD11b+ cells of the stroma. Small molecular weight cytokines with chemoattractant properties, chemokines, have a critical role in regulating APC migration and activation. Immature dendritic cells (i.e., APCs) express the chemokine receptors CCR1, CCR2, and CCR5, which mediate mobilization to sites of inflammation. By contrast, mature APCs up-regulate their expression of CCR7, which facilitates homing to secondary lymphoid tissues.

As described in detail below, the expression of chemokine receptors CCR1, CCR2, CCR5, and CCR7 by corneal CD11b+ APCs in DED was characterized. The mRNA expression of chemokine ligands CCL2 (MCP-1), CCL4 (MIP-1β), and CCL5 (RANTES) at the ocular surface was quantified. Finally, the dynamics of homing of mature CCR7+CD11b+ APCs into draining lymph nodes in DED was investigated.

DED was induced by exposing 6-8 week female C57BL/6 mice to desiccating stress within a controlled environment chamber and by administering daily scopolamine injections. Age and gender matched mice were housed within the normal environment of the animal facility and used as controls. Mice were sacrificed at day 12.

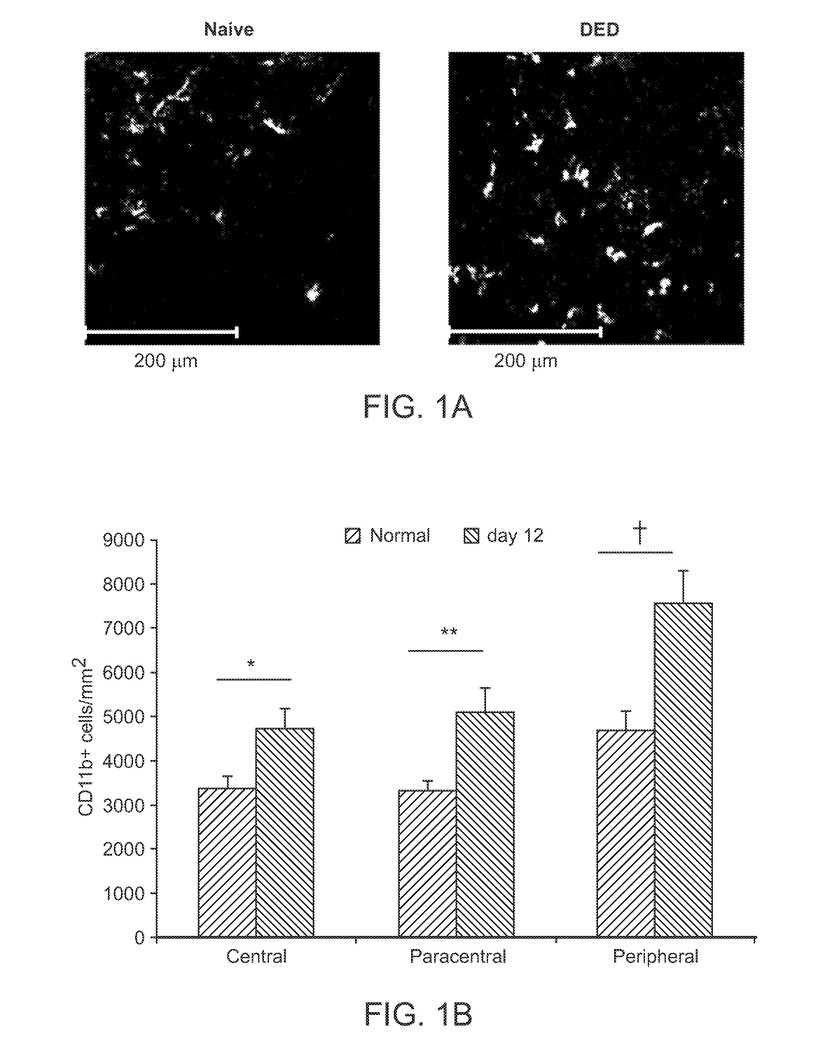

For whole-mount immunohistochemical staining, freshly harvested corneas were fixed with acetone and stained with CD11b in combination with either CCR1, CCR2, CCR5, or CCR7 and analyzed using confocal microscopy. The results of enumeration of CD11b+ cells in the cornea are shown in FIG. 1. FIG. 1A shows representative confocal images of whole-mount corneas stained with CD11b-Alexa 488 (Magnification ×400). FIG. 1B demonstrates the enumeration of CD11b+ cells showing significantly increased numbers of CD11b+ cells in the corneal stroma of DED mice. P values have been determined using the student T test and error bars represent SEM. *, p=0.02, **, p<0.01, †, p<0.001.

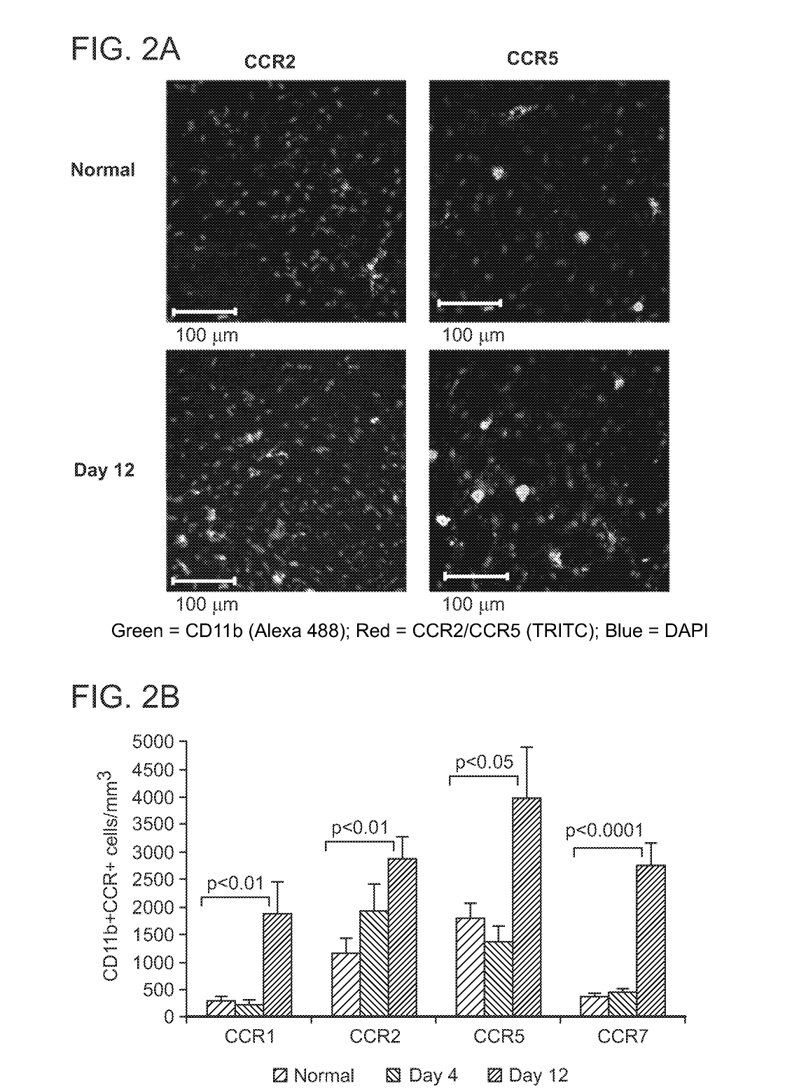

FIG. 2 illustrates the results of enumeration of chemokine receptor expressing CD11b+ cells in the cornea. FIG. 2A shows representative confocal images of whole-mount corneas double stained with CD11b and either CCR2 or CCR5 (Magnification ×400). As shown in FIG. 2B, increased frequencies of CCR1, CCR2, CCR5, and CCR7 expressing CD11b+ cells were observed in the DED corneal stroma at day 12.

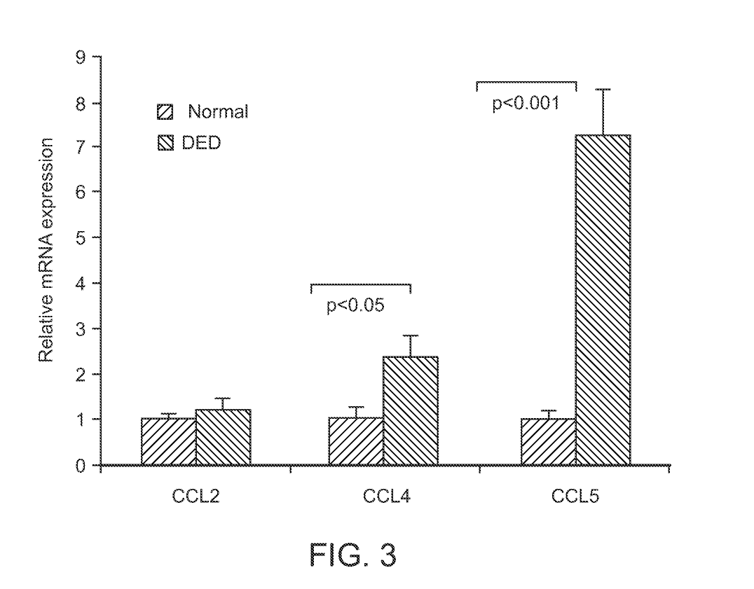

Reverse transcriptase polymerase chain reaction (RT-PCR) was performed on corneal and conjunctival tissue to quantity the relative messenger ribonucleic acid (mRNA) expression of the chemokine ligands CCL2, CCL4, and CCL5. The relative mRNA expression of chemokine ligands at the ocular surface is shown in FIG. 3. Real-time RT PCR analysis showed a significant increase in the relative mRNA expression of CCL4 and CCL5 in conjunctival tissue from DED mice. A similar trend of elevated chemokine mRNA transcript levels was observed within the cornea of DED mice.

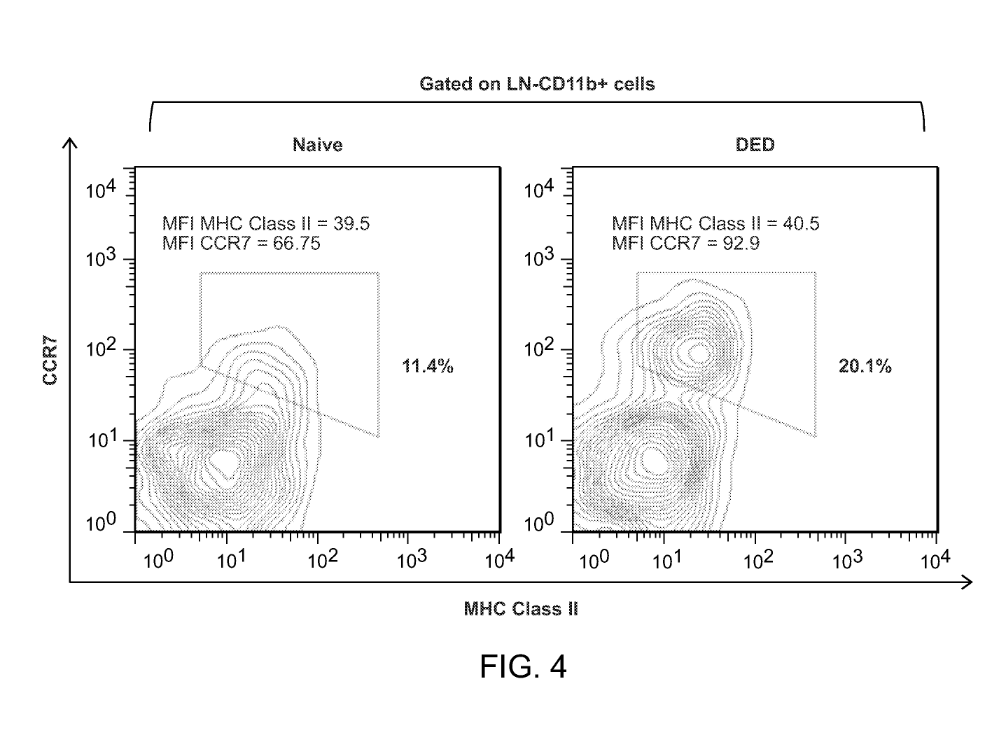

Finally, flow cytometric analysis was carried out on lymph node cells pooled from regional lymph node tissue, and triple stained for CD11b, MHC Class II, and CCR7. The frequencies of CCR7 expressing mature (MHC Class II+) CD11b+ APCs in draining cervical lymph nodes from normal and DED mice is illustrated in FIG. 4. Specifically, representative flow cytometric analysis showed increased homing of CCR7+ mature CD11b+ APCs to the draining lymph nodes of mice with DED compared with normal mice (FIG. 4).

Thus, the results presented herein demonstrate the following in this model of dry eye disease: increased frequencies of CD11b+ cells within the corneal stroma, up-regulation of chemokine receptors CCR1, CCR2, CCR5, and CCR7 by corneal CD11b+ cells, elevated expression of chemokine ligands CCL4 and CCL5 at the ocular surface, and increased homing of CCR7+ mature APCs in draining lymph nodes.

The data indicate that chemokine-mediated mechanisms of cell trafficking have an important function in the initiation of the immune response in DED, by influencing both the migration of APCs to the ocular surface, and subsequent homing of these cells to draining lymphoid tissues.

### Example 2

### Topical CCR7 Blockade is Highly Effective in Inhibiting the Immunopathogenesis of DED

The critical contribution of inflammation to the development and progression of DED has become increasingly clear in recent years. Hyperosmolarity resulting from desiccating stress induces the epithelial expression of pro-inflammatory cytokines and chemokines. Chemokine-mediated recruitment of immature antigen-presenting cells (APCs) to the ocular surface ensues (as explained above), with the pro-inflammatory milieu promoting their subsequent activation and maturation (Stevenson et al., 2012 Arch Ophthalmol, 130:90-100). In accordance with the acquisition of maturation markers, mature APCs up-regulate the chemokine receptor CCR7, which facilitates their directional migration towards lymphoid tissue, in response to a CCL21 chemotactic gradient generated in part by the lymphatic endothelium (Saeki et al., 1999 J Immunol, 162: 2472-2475). By capture and subsequent presentation of Antigen to T helper (Th) lymphocytes, as well as being involved in driving T cell differentiation (e.g., Th1, Th2, and Th17), APCs play a central role in the induction of adaptive immune responses. Unparalleled expression of MHC and costimulatory molecules (e.g., B7.1 and B7.2) allows mature APCs to be potent T cell stimulators.

As described above, there was an increase in the frequencies of CCR7 expressing mature APCs in the regional lymph nodes (LN) of DED mice (FIG. 4). In the regional LNs, APCs activate IL-17 producing T-helper cells (Th17), which have been identified as the dominant effectors in DED and capable of corneal epithelial barrier disruption.

Described in detail below is the prevention of the induction and maintenance of Th17 mediated immunity in DED by interfering with the trafficking of mature APCs from the ocular surface to regional LNs through the topical blockade of CCR7. This approach has been successfully implemented by Schlereth who observed significantly reduced clinical scores following topical treatment with anti-CCR7 antibody in their model of allergic conjunctivitis (Schlereth et al., 2012 Am J Pathol, 180: 2351-60).

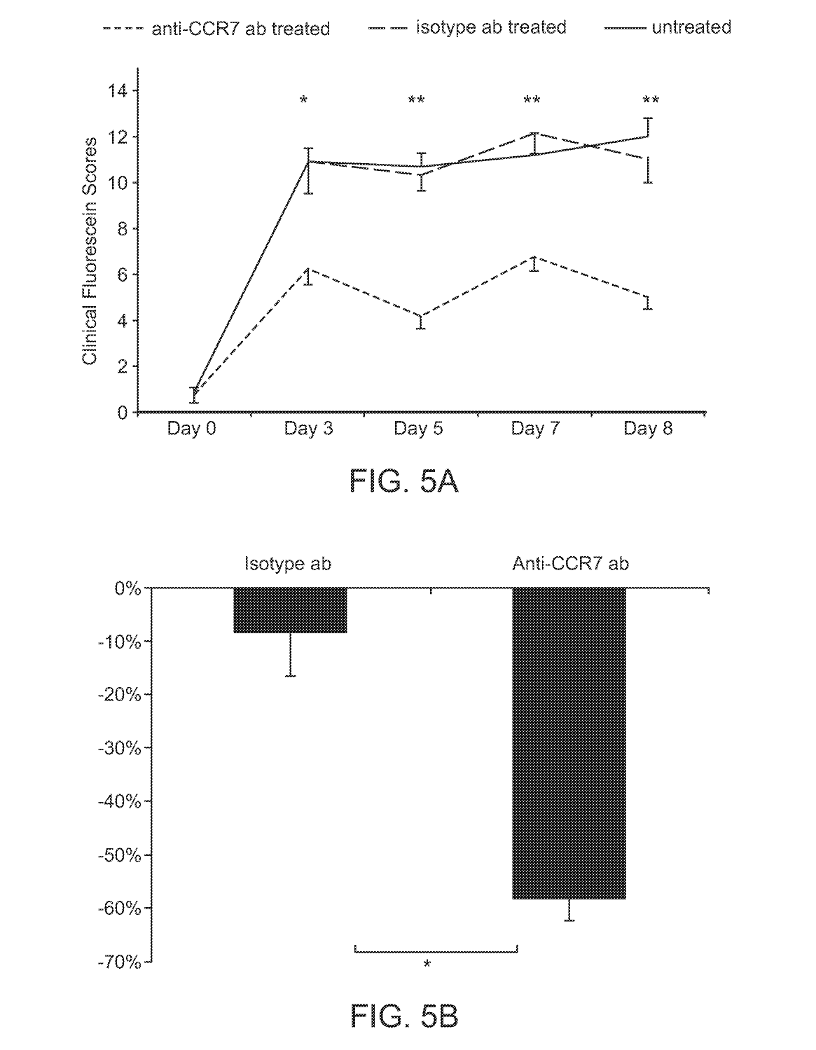

DED was induced by exposing mice to a desiccating environment within a controlled environment chamber (CEC), and supplementing this with subcutaneous injections of scopolamine. Initially, the role of topical CCR7 blockade on the induction of DED and Th17 immunity was examined. Mice were treated topically from the first day of exposure to the CEC with either anti-CCR7 antibody (clone 4B12; R&D Systems, catalog number: MAB3477), isotype control antibody (R&D Systems, catalog number: MAB006) or remained untreated. The anti-CCR7 antibody was administered at a concentration of 1% (mg/ml) using a volume of 2 μl/dose instilled topically onto the ocular surface. The clinical severity of the disease was evaluated by clinical fluorescein scoring (CFS) using the National Eye Institute (NEI) grading scheme. A significant difference (p<0.0001) in CFS scores were observed between the anti-CCR7 and isotype treated groups over the 8 days of observation (FIG. 5A). FIG. 5B shows the mean percentage reduction in CFS scores (normalized to mean CFS scores in untreated DED group) in isotype and anti-CCR7 treated mice at day 8.

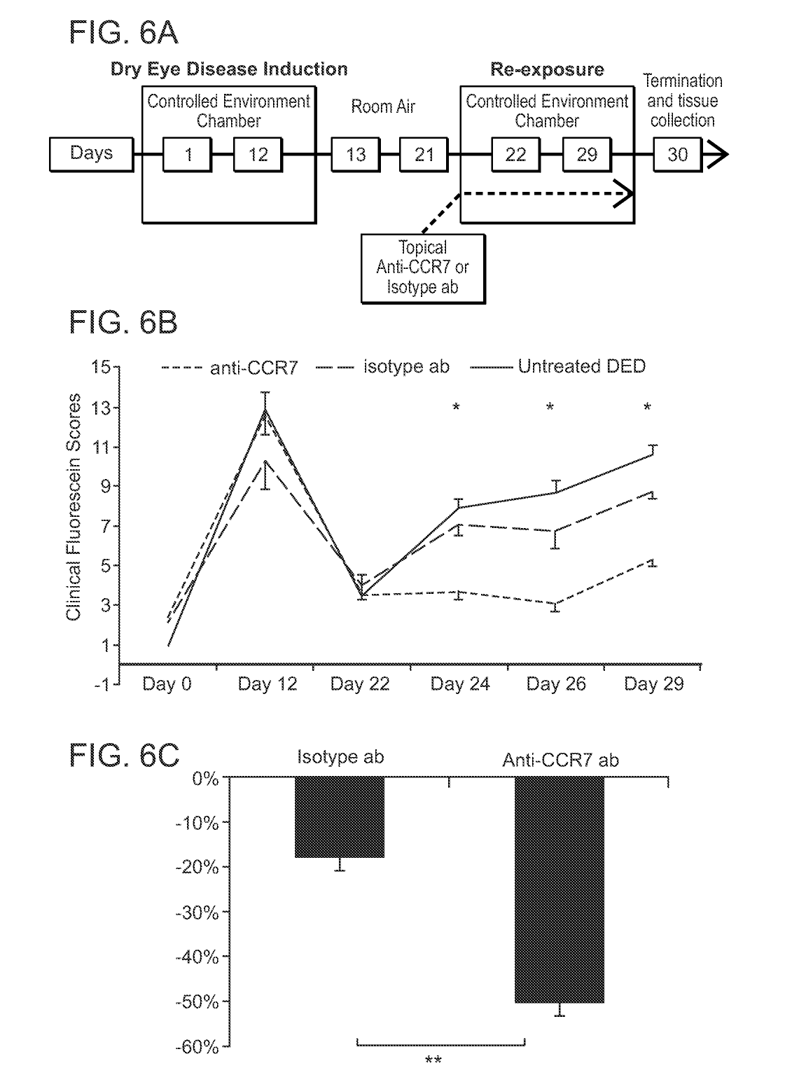

Next, the effect of topical CCR7 blockade in previously sensitized mice that were re-exposed to the desiccating environment of the CEC for a second time was examined. The purpose of the second experiment (FIG. 6) was to evaluate the potential therapeutic value of this approach in chronic DED by determining the effect of topical CCR7 blockade on DED in mice that had been re-exposed to the CEC following prior induction of DED. Dry eye was initially induced as outlined above for a total of 12 days. Mice from all 3 groups were initially placed into the CEC until day 12, with the subsequent observed rise in CFS scores confirming the primary induction of DED. The DED mice were then removed from the CEC and housed in room air until day 22, during which time CFS scores decreased, but remained higher than baseline. During the induction and room air phases, all mice remained untreated. At day 22, mice were returned to the CEC, and treatment commence in all 3 groups. Because treatment began on day 22, the therapeutic effect of blockade of CCR7 was observed from this time point forward.

A schematic diagram of the experimental design to study the effect of topical CCR7 blockade on chronic DED is shown in FIG. 6A. A similar difference (p<0.001) in CFS scores was observed in the anti-CCR7 treated group compared to isotype treated (FIG. 6B), suggesting that topical CCR7 blockade is effective in ameliorating DED following prior sensitization. FIG. 6C shows the mean percentage reduction in CFS scores (normalized to mean CFS scores in untreated DED group) in isotype and anti-CCR7 treated mice at day 29. **, p=0.0001.

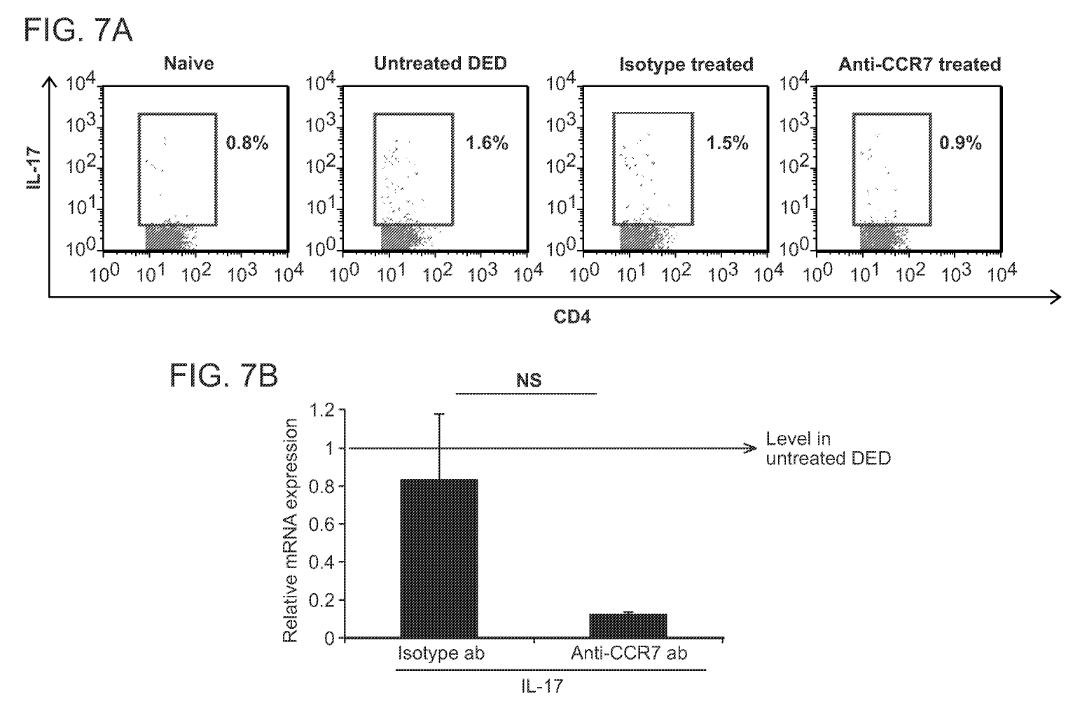

In order to examine the effect on Th17 induction, frequencies of CD4+IL-17+ cells in the regional draining lymph nodes of the mice utilized in the experiments above were quantified using flow cytometry. A reduction in Th17 frequencies was demonstrated in the anti-CCR7 treated group (0.9%), compared to isotype treated (1.5%) and untreated (1.6%), suggestive of a minimal Th17 response in the draining lymph node of anti-CCR7 treated mice (FIG. 7A). These findings are in accord with the reduction of IL-17a expression observed in the conjunctiva of anti-CCR7 DED mice compared to isotype treated mice (FIG. 7B).

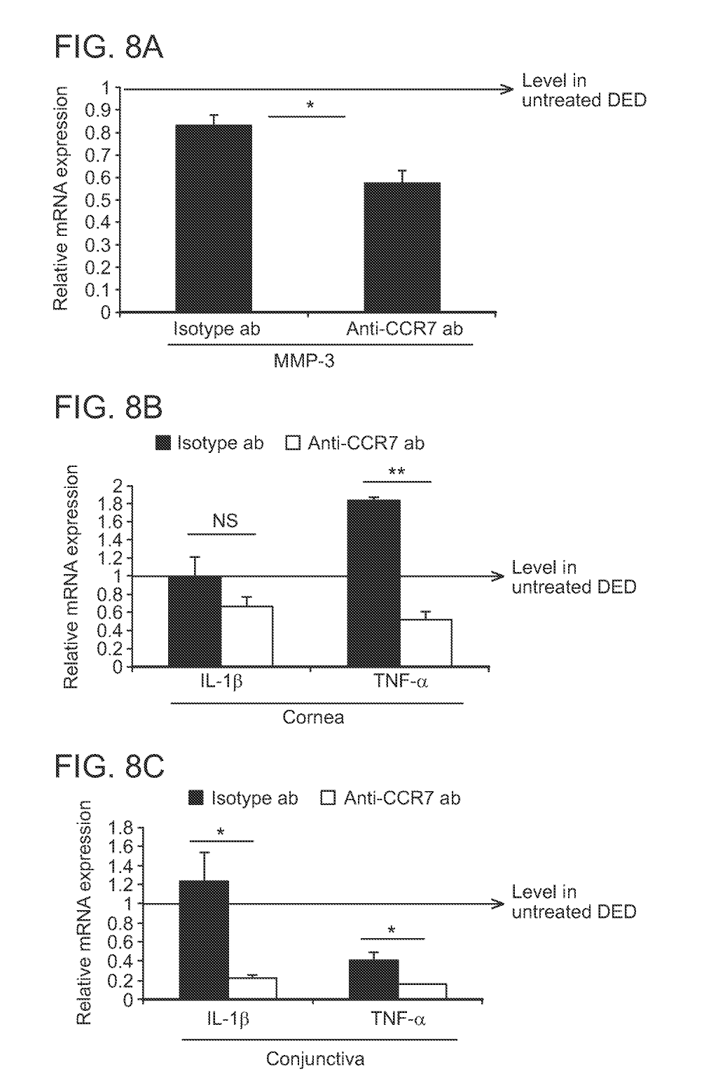

Next, conjunctival mRNA expression of pro-inflammatory cytokines was determined. Since IL-17 has been implicated in inducing DED associated up-regulation of matrix metalloproteinase-3 (MMP-3) which are critical to corneal epithelial barrier disruption (Chauhan and Dana, 2009 Mucosal Immunol, 2:243-253), mRNA expression of MMP-3 in corneal tissue was determined. Decreased mRNA transcript levels of MMP-3, (p<0.05), was demonstrated in anti-CCR7 treated corneas (FIG. 8A). Decreased expression levels of TNF-α and IL-1β were observed in conjunctival and corneal tissue derived from anti-CCR7 treated mice, compared to isotype treated (FIG. 8B and FIG. 8C). Values presented are the mean of three. N=3-6 mice per group.

In summary, the data presented herein demonstrates that topical CCR7 blockade is highly effective in inhibiting the immunopathogenesis of DED and implicates the contribution of CCR7 mediated trafficking of mature APCs in driving the induction and maintenance of the Th17 response in DED. Thus, topical CCR7 antagonism is an effective therapeutic strategy for the treatment of DED and ocular surface disorders.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of treating ocular surface inflammatory disease comprising:
identifying a subject who has been diagnosed with an ocular surface inflammatory disease; and
administering to an ocular or adnexal tissue a composition comprising an effective amount of a C-C chemokine receptor type 7 (CCR7) inhibitor.

2. The method of claim 1, wherein said ocular surface inflammatory disease is selected from the group consisting of dry eye disease, Stevens-Johnson syndrome, and microbial keratitis.

3. The method of claim 2, wherein said dry eye disease comprises keratoconjunctivitis sicca (KCS), Sj [symbol] gren's syndrome (SS), Sj [symbol] gren's syndrome associated keratoconjunctivitis sicca, non-Sj [symbol] gren's syndrome associated keratoconjunctivitis sicca, keratitis sicca, sicca syndrome, xerophthalmia, tear dysfunction disorder, decreased tear production, aqueous tear deficiency (ATD), meibomian gland dysfunction, exposure keratopathy, or hyperevaporate tear deficiency.

4. The method of claim 1, wherein said CCR7 inhibitor comprises a composition that inhibits or modifies the function, transcription, transcription stability, translation, modification, localization, or secretion of a polynucleotide or polypeptide encoding CCR7 or a CCR7 associated ligand, wherein said CCR7 associated ligand is CCL19 or CCL21.

5. The method of claim 1, wherein said CCR7 inhibitor is selected from the group consisting of an anti-CCR7 antibody, a small molecule antagonist of CCR7, a peptide that blocks CCR7, a blocking fusion protein of CCR7, an anti-CCL19 antibody, or an anti-CCL21 antibody.

6. The method of claim 5, wherein said anti-CCR7 antibody has a binding specificity to CCR7, CCL19, or CCL21 in the species of said subject.

7. The method of claim 6, wherein said neutralizing antibody is a monoclonal antibody, a polyclonal antibody, a single chain antibody, a humanized antibody, a recombinant antibody, or a chimeric antibody.

8. The method of claim 1, wherein said CCR7 inhibitor comprises an antibody conjugated directly or indirectly to a compound that inhibits or modifies the activity of CCR7.

9. The method of claim 1, wherein said CCR7 inhibitor is administered at a dose effective to reduce or prevent migration of antigen-presenting cells to lymphoid tissue of said subject.

10. The method of claim 1, wherein said CCR7 inhibitor is administered at a dose effective to reduce or prevent the induction or maintenance of a pro-inflammatory T helper 1 (Tₕ1) and T helper 17 (Tₕ17) response in the draining lymphoid tissue of a subject, leading to a reduction of Tₕ17-mediated immunity in an ocular or adnexal tissue in said subject.

11. The method of claim 1, wherein said composition is administered topically or subconjunctivally.

12. The method of claim 1, wherein said composition is administered onto said ocular surface.

13. The method of claim 1, further comprising the administration of a pharmaceutically acceptable carrier, one or more tear substitute(s), or an ophthalmic lubricant.

14. The method of claim 1, further comprising the administration of a second therapeutic agent.

15. The method of claim 14, wherein said second therapeutic agent comprises a corticosteroid, cyclosporine, an agent that targets interleukin-1 (IL-1), an agent that targets interleukin-17 (IL-17), an agent that targets tumor necrosis factor alpha (TNF-α), or an agent that targets matrix metalloproteinase-3 (MMP-3).

16. (canceled)

17. (canceled)

18. The method of claim 1, wherein said subject is a mammal.

19. The method of claim 18, wherein said mammal is a human.

20. The method of claim 1, wherein said CCR7 inhibitor is administered topically at a dose of 1 to 2 drops or subconjunctivally at a dose of 0.5-1 ml.

21. The method of claim 1, wherein said CCR7 inhibitor is present in a concentration of 0.001-10% (mg/ml).

22. The method of claim 1, wherein said composition is in the form of a solid, a paste, an ointment, a gel, a liquid, an aerosol, a mist, a polymer, a film, an emulsion, a depot preparation, a suspension, or incorporated into or coated onto a contact lens.

23. (canceled)

24. The method of claim 1, wherein said CCR7 inhibitor is administered every 72 hours, every 48 hours, every 24 hours, every 12 hours, every 6 hours, every 3 hours, or every 1 hour.

25. The method of claim 1, wherein said CCR7 inhibitor is administered for 3 days, 7 days, 14 days, 30 days, 60 days, 90 days, 120 days, 150 days, 180 days, 210 days, 240 days, 270 days, 300 days, 330 days, or 360 days.

26. The method of claim 1, wherein levels of tumor necrosis factor alpha (TNF-α), interleukin-17 (IL-17), IL-1β, or matrix metalloproteinase-3 (MMP-3) are reduced.

27. (canceled)

28. The method of claim 9, wherein said antigen-presenting cells include CD11b+ cells.

29. A method of reducing or preventing migration of antigen-presenting cells to lymphoid tissue comprising administering to ocular or adnexal tissue a composition comprising a CCR7 inhibitor.

30. A method of reducing or preventing the induction of pro-inflammatory T helper 1 (Tₕ1) and T helper 17 (Tₕ17)-mediated immunity in the draining lymphoid tissue of a subject with a resulting decrease in Tₕ17-mediated immunity in an ocular or adnexal tissue in a subject, said method comprising administering to said ocular or adnexal tissue a composition comprising a CCR7 inhibitor in an amount effective to decrease a population of Tₕ17 cells in said ocular or adnexal tissue, thereby reducing or preventing the induction of pro-inflammatory T helper 17 (Tₕ17)-mediated immunity.

31. A method of preventing ocular surface inflammatory disease comprising:
identifying a subject who is at risk for developing an ocular surface inflammatory disease; and
administering to an ocular or adnexal tissue a composition comprising an effective amount of a C-C chemokine receptor type 7 (CCR7) inhibitor.

32. The method of claim 31, wherein said subject has undergone refractive surgery.

33. The method of claim 31, wherein said ocular surface inflammatory disease is selected from the group consisting of dry eye disease, Stevens-Johnson syndrome, and microbial keratitis.

34. A composition comprising a C-C chemokine receptor type 7 (CCR7) inhibitor for treating an ocular surface inflammatory disease in a subject, wherein said composition is administered to an ocular or adnexal tissue of the subject.

35. The composition of claim 34, wherein said ocular surface inflammatory disease is selected from the group consisting of dry eye disease, Stevens-Johnson syndrome, and microbial keratitis.

36. The composition of claim 35, wherein said dry eye disease comprises keratoconjunctivitis sicca (KCS), Sj [symbol] gren's syndrome (SS), Sj [symbol] gren's syndrome associated keratoconjunctivitis sicca, non-Sj [symbol] gren's syndrome associated keratoconjunctivitis sicca, keratitis sicca, sicca syndrome, xerophthalmia, tear dysfunction disorder, decreased tear production, aqueous tear deficiency (ATD), meibomian gland dysfunction, exposure keratopathy or hyperevaporate tear deficiency.

37. The composition of claim 34, wherein said CCR7 inhibitor comprises a composition that inhibits or modifies the function, transcription, transcription stability, translation, modification, localization, or secretion of a polynucleotide or polypeptide encoding CCR7 or a CCR7 associated ligand, wherein said CCR7 associated ligand is CCL19 or CCL21.

38. The composition of claim 34, wherein said CCR7 inhibitor is selected from the group comprising an anti-CCR7 antibody, a small molecule antagonist of CCR7, a peptide that blocks CCR7, a blocking fusion protein of CCR7, an anti-CCL19 antibody, or an anti-CCL21 antibody.

39. The composition of claim 38, wherein said anti-CCR7 antibody has a binding specificity to CCR7, CCL19, or CCL21.

40. The composition of claim 39, wherein said antibody is a monoclonal antibody, a polyclonal antibody, a single chain antibody, a humanized antibody, a recombinant antibody, or a chimeric antibody.

41. The composition of claim 34, wherein said CCR7 inhibitor comprises an antibody conjugated directly or indirectly to a compound that inhibits or modifies the activity of CCR7.

42. The composition of claim 34, wherein said composition is administered at a dose effective to reduce or prevent migration of antigen-presenting cells to lymphoid tissue of said subject.

43. The composition of claim 34, wherein said composition is administered at a dose effective to reduce or prevent the induction or maintenance of a pro-inflammatory T helper 1 (Th1) and T helper 17 (Th17) response in the draining lymphoid tissue of said subject, leading to a reduction of Th17-mediated immunity in an ocular or adnexal tissue in said subject.

44. The composition of claim 34, wherein said composition is administered topically or subconjunctivally.

45. The composition of claim 34, further comprising a second therapeutic agent.

46. The composition of claim 45, wherein said second therapeutic agent comprises a corticosteroid, cyclosporine, an agent that targets interleukin-1 (IL-1), an agent that targets interleukin-17 (IL-17), an agent that targets tumor necrosis factor alpha (TNF-α), or an agent that targets matrix metalloproteinase-3 (MMP-3).

47. The composition of claim 34, further comprising one or more tear substitutes, or an ophthalmic lubricant.

48. The composition of claim 34, wherein said CCR7 inhibitor is present in a concentration of 0.001-10% (mg/ml).

49. The composition of claim 34, wherein said composition is in the form of a solid, a paste, an ointment, a gel, a liquid, an aerosol, a mist, a polymer, a film, an emulsion, a depot preparation, or a suspension.

50. The composition of claim 34, wherein levels of tumor necrosis factor alpha (TNF-α), interleukin-17 (IL-17), IL-1β, or metalloproteinase (MMP-3) are reduced.

51. A pharmaceutical composition comprising the composition of claim 34 and a pharmaceutically acceptable carrier.
